(19) United States
(12) Patent Application Publication
(10) Pub. No.: US 2026/0137298 A1
(43) Pub. Date: May 21, 2026

(54) DELIBERATE RECIPIENT CREATION OF BIOLOGICAL ENVIRONMENT

(71) Applicant: Cochlear Limited, Macquarie University, NSW (AU)

(72) Inventors: Kostas Ioannis TSAMPAZIS, North Ryde, NSW (AU); Helmut Christian EDER, Bemboka, NSW (AU); Dylan WHITEHOUSE, Sydney, NSW (AU); Padraig HURLEY, Frenches Forest, NSW (AU)

(21) Appl. No.: 19/118,908

(22) PCT Filed: Oct. 4, 2023
(86) PCT No.: PCT/IB2023/059966

§ 371 Date: Apr. 7, 2025

Related U.S. Application Data
(60) Provisional application No. 63/415,854, filed on Oct. 13, 2022.

(51) Int. Cl. A61B 5/08; A61B 5/00; A61B 5/283; A61B 7/00; A61B 7/04

(52) U.S. Cl. CPC A61B 5/0803; A61B 5/283; A61B 5/686; A61B 5/7246; A61B 5/7405; A61B 5/742; A61B 5/746; A61B 7/003; A61B 7/04; A61B 2560/0468; A61B 2562/0204

(57) ABSTRACT

Presented herein are techniques for prompting a recipient to deliberately create a biological environment suitable for one or more biological measurements. In one aspect, a medical device generates a notification to prompt a recipient to consciously execute a specified biological event, and, while the specified biological event is occurring, monitors a bodily function of the recipient. In another aspect, the medical device prompts a recipient to produce a particular biological environment, and detects, in the particular biological environment, one or more sound signals associated with a target bodily function of the recipient. In another aspect, the medical device outputs an instruction for a recipient to create a specified biological state of the recipient, and stores one or more sound signals generated during the specified biological state of the recipient for diagnosis of the recipient with a medical condition.

### BACKGROUND

### Field of the Invention

The present invention relates generally to prompting a recipient to deliberately create a biological environment suitable for one or more biological measurements.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a first method is provided. The first method comprises: generating a notification to prompt a recipient to consciously execute a specified biological event; determining that the specified biological event is occurring; and while the specified biological event is occurring, monitoring a bodily function of the recipient.

In another aspect, a medical device is provided. The medical device comprises: one or more processors configured to prompt a recipient to produce a particular biological environment; and a microphone configured to detect, in the particular biological environment, one or more sound signals associated with a target bodily function of the recipient.

In another aspect, a second method is provided. The second method comprises: outputting an instruction for a recipient to create a specified biological state of the recipient; monitoring one or more sound signals generated during the specified biological state of the recipient; and storing the one or more sound signals generated during the specified biological state of the recipient for diagnosis of the recipient with a medical condition.

### BRIEF DESCRIPTION OF THE DRAWINGS

Embodiments of the present invention are described herein in conjunction with the accompanying drawings, in which:

FIG. 1 is a block diagram illustrating an implantable medical device with which aspects of the techniques presented herein can be implemented;

FIG. 2A is a block diagram illustrating a first cochlear implant system with which aspects of the techniques presented herein can be implemented;

FIG. 2B is a block diagram illustrating a second cochlear implant system with which aspects of the techniques presented herein can be implemented;

FIG. 3 is a schematic diagram illustrating a vestibular stimulator system with which aspects of the techniques presented herein can be implemented;

FIG. 4 is a schematic diagram illustrating a retinal prosthesis system with which aspects of the techniques presented herein can be implemented;

FIG. 5 is a flowchart of an example method, in accordance with certain embodiments presented herein;

FIG. 6 is another flowchart of an example method, in accordance with certain embodiments presented herein; and

FIG. 7 is another flowchart of an example method, in accordance with certain embodiments presented herein.

### DETAILED DESCRIPTION

Presented herein are techniques for prompting a recipient to deliberately create a biological environment suitable for one or more biological measurements. In one example, a medical device is provided which can prompt a recipient to consciously execute a specified biological event and monitor a bodily function of the recipient, via a microphone, while the specified biological event is occurring. The specified biological event can establish conditions suitable for collecting high-quality body sound recordings (e.g., heart or lung sounds with high signal-to-noise ratios and minimal interference).

The medical device can achieve sufficient signal-to-noise ratio while being sufficiently insensitive to biological/body noises. The biological/body noises are undesirable sounds induced by the body that are propagated primarily as vibration, such as breathing, scratching, rubbing, noises associated with the movement of the head, chewing, own voice (i.e., when the recipient speaks), etc. Conventional medical devices typically collect relatively poor diagnostic audio data that includes the body noises and therefore has a relatively low signal-to-noise ratio. For instance, in conventional approaches, audio signals generated by the heart can be distorted by the recipient's breathing, or a recipient is unable to breathe deeply enough to produce useful audio signals generated by the lungs.

The medical device described herein can include heart and lung monitoring and diagnostic capabilities and functions that utilize heart and lung sounds. In one example, recordings can be arranged to coincide with conscious breathing events of the recipient. For instance, initiation of a lung health recording can be arranged to coincide with the end of a deep breath. Similarly, initiation of a heart health recording can be arranged to coincide with the holding of a conscious breath. The recipient can be prompted to breathe deeply (or hold their breath) in a range of ways, including via auditory instructions or other examples.

Unlike traditional medical devices (e.g., conventional pacemakers), the medical device described herein can detect and analyze heart and lung sounds for heart and lung issues (diseases) for diagnostic and prevention purposes-that is, to detect and help prevent heart/lung issues. In one example, the medical device can be a cochlear implant configured to perform diagnostics on, and provide insights regarding, heart and lung health, thereby serving as an early detector of abnormalities of heart and lung function.

The medical device can detect any suitable measurement, such as heart beats, heart rate variations, breathing variations, heart/lung sound, sound distortion, etc. The sound distortion in particular can indicate heart/lung issues in an early stage, even before heart/breathing variations occur. The high-quality measurements collected by the medical device can help monitor the heart and lung health of a recipient by providing early detection/diagnosis of heart and lung issues. Furthermore, improved quality and convenience of the recordings can be suitable for general cardiovascular health monitoring of wide population groups. Heart and lung diagnostics can be carried out automatically by the medical device, an external device, a server (e.g., as a Software-as-a-Service (Saas), etc. The diagnostics can also be performed manually (e.g., by a medical professional) or semi-automatically (e.g., where at least some data is processed/pre-processed autonomously and analyzed manually).

In one example, as referenced above, a microphone of the medical device can monitor and record audio signals from the heart and lungs to be used for providing diagnostic services on heart and lung health (e.g., monitoring performance, detecting abnormalities, etc.). It will be appreciated that the techniques described herein can apply to any suitable medical diagnostics, and are not necessarily limited to diagnostics of the heart and lungs. For instance, audio signals detected by a microphone, such as an implantable microphone, can be used to measure changes in acoustics that can signal concussions, among other medical issues.

In certain examples, one or more relevant cardiovascular measures (e.g., biopotentials) can also be collected/monitored/analyzed using one or more electrodes to assist in the heart and lung diagnostics. The audio recording and biopotential measurement can be taken simultaneously and correlated/combined to further to sense and detect heart/lung issues more precisely and earlier. Biopotentials can also be monitored before the audio recording begins for the purpose of scheduling an audio recording with conscious recipient participation (e.g., deliberate breathing).

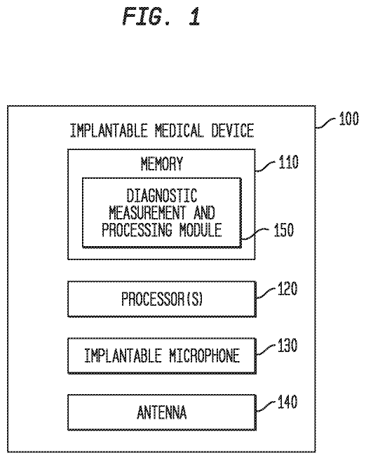

FIG. 1 is a block diagram illustrating an implantable medical device 100 with which aspects of the techniques presented herein can be implemented. Implantable medical device 100 can be any suitable implantable medical device. In one specific example, implantable medical device 100 can be a cochlear implant system. However, it is to be appreciated that implantable medical device 100 can also be other types of implantable medical devices. For example, implantable medical device 100 can be an auditory prosthesis system that includes one or more types of auditory prostheses, such as middle ear auditory prostheses, bone conduction devices, direct acoustic stimulators, electro-acoustic prostheses, auditory brain stimulators, combinations, or variations thereof, etc. Implantable medical device 100 can also be a dedicated tinnitus therapy device. In further embodiments, implantable medical device 100 can also be a vestibular device (e.g., vestibular implant), visual device (i.e., bionic eye), sensor, pacemaker, drug delivery system, defibrillator, functional electrical stimulation device, catheter, seizure device (e.g., device for monitoring and/or treating epileptic events), sleep apnea device, electroporation device, etc.

Implantable medical device 100 includes memory 110, one or more processors 120, implantable microphone 130, and antenna 140. Memory 110 can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. One or more processors 120 are, for example, microprocessors or microcontrollers that execute instructions stored in memory 110.

Implantable microphone 130 can be configured to detect input (sound/vibration) signals (e.g., body noises) and convert the detected input signals into electrical signals. In one example, implantable microphone 130 can be a wide-frequency-bandwidth microphone capable of sensing and recording the frequency spectrum of the heart and lung sound signals. Antenna 140 can enable the transfer of power and/or data between implantable medical device 100 and a corresponding external device.

As shown, memory 110 includes diagnostic measurement and processing module (e.g., logic) 150. Briefly, diagnostic measurement and processing module 150 can cause processor(s) 120 to perform techniques described herein with respect to monitoring bodily functions in a deliberately created biological environment. In one example, processor(s) 120 can generate or output a notification/instruction/command to prompt a recipient to consciously execute a specified biological event/state and thereby produce a particular biological environment. Like diagnostic measurement and processing module 150, the instruction(s) can be stored in and retrieved from memory 110.

Processor(s) 120 can determine that the specified biological event is occurring and, while the specified biological event is occurring, monitor a bodily function of the recipient. To that end, implantable microphone 130 can be configured to detect, in the particular biological environment, one or more sound signals associated with a target bodily function of the recipient. Processor(s) 120 can monitor the sound signals generated during the specified biological state and store them for diagnosis of the use with a medical condition (e.g., a heart or lung issue). Therefore, implantable medical device 100 can act as a respiration and/or cardiac sensor configured to detect respiration and/or cardiac signals used for lung and/or heart monitoring/diagnosis.

In one example, the notification can prompt the recipient to temporarily cease breathing (e.g., hold their breath), and while the recipient has temporarily ceased breathing, processor(s) 120 can record heart activity of the recipient (e.g., using implantable microphone 130). When breathing has ceased, the heart sound can be recorded in high quality (e.g., without noise caused by breathing).

In another example, the notification can prompt the recipient to breathe deeply, and while the recipient is breathing deeply, processor(s) 120 can record lung activity of the recipient (e.g., using implantable microphone 130). The deep breathing can yield high-quality lung sound recordings. To obtain the lung activity of the recipient while minimizing or eliminating interference on the lung sound signal due to noise generated by the heart, processor(s) 120 can monitor a first sound signal corresponding to the heart activity of the recipient (e.g., using another microphone), record a second sound signal while the recipient is breathing deeply, and subtract the first sound signal from the second sound signal.

Implantable medical device 100 can generate the notification in response to any suitable stimulus. In one example, implantable medical device 100 can generate the notification in response to obtaining, from the recipient, an indication to monitor the bodily function of the recipient. The recipient can activate the diagnostic functions of implantable medical device 100 on-demand via an external device such as a recipient device or an external component corresponding to the implantable medical device. The external device can be a computing device such as a computer (e.g., laptop, desktop, tablet), a mobile phone, remote control unit, etc.

In another example, implantable medical device 100 can generate the notification in response to one or more triggering biological events of the recipient. Examples of triggering biological events can include the lungs being fully inhaled/exhaled, the recipient holding their breath, etc. In still another example, implantable medical device 100 can generate the notification at a predetermined time (e.g., periodically, such as at the same time every day).

Implantable medical device 100 (e.g., in combination with the external device) can provide the notification as an auditory prompt and/or a visual prompt for the recipient to consciously execute the specified biological event. As a result, the recipient can participate in the process of heart/lung diagnostic procedure based on initial and feedback audio instructions in real-time. For example, the external device can generate an auditory prompt via a speaker and/or a visual prompt via a display (e.g., a screen). An auditory prompt can be an instruction such as, “Take a deep breath and, after the beep, hold your breath for ten seconds or until the next beep. Don't move during the test” or “After the beep, breathe deeply for ten seconds or until the next beep.” A visual prompt can be an instruction such as “Take a deep breath and, after the text “Hold Breath” appears, hold your breath for ten seconds or until the text disappears. Don't move during the test.” or “After the text “Hold Breath” appears, breathe deeply for ten seconds or until the text disappears.” After the test, implantable medical device 100 can determine whether the measurement is acceptable. If not, implantable medical device 100 can provide a further auditory or visual prompt to repeat the measurement (e.g., “The test is repeating . . . ”). Or, if the measurement is acceptable, implantable medical device 100 can provide a further auditory or visual prompt such as “Test completed.”

Implantable medical device 100 can determine that the specified biological event is occurring (e.g., that the recipient has complied with the auditory/visual prompt) in any suitable manner. In one example, implantable medical device 100 can determine that a predefined period of time after generating the notification has expired. For instance, implantable medical device 100 can generate the notification, wait five seconds (for instance), and then proceed to take the measurement. In another example, implantable medical device 100 can obtain, from the recipient, an explicit indication that the recipient has executed the specified biological event. For instance, the recipient can select a button on a smartphone or remote control indicating that they are breathing deeply or holding their breath. In still another example, implantable medical device 100 can obtain a biological indication that the recipient has executed the specified biological event. For instance, implantable microphone 130 can detect sound signals indicating that the recipient is breathing deeply or holding their breath.

Processor(s) 120 can be configured to adjust a gain of implantable microphone 130 (e.g., an amplifier of implantable microphone 130) for implantable microphone 130 to detect the one or more sound signals associated with the target bodily function of the recipient. For example, when implantable medical device 100 determines that the specified biological event is occurring, processor(s) 120 can increase the gain of implantable microphone 130 to improve sound signal detection. By increasing the gain, the sensitivity of the heart and lung recordings can be improved.

Processor(s) 120 can compare the sound signals generated during the specified biological state with one or more historical sound signals (e.g., sound signals that were previously generated during the specified biological state). For example, processor(s) 120 can compare (a) the sound signals obtained when a recipient is holding their breath to (b) stored sound signals previously obtained when the recipient held their breath, and identify any changes in the sound signals relative to the historical sound signals.

Processor(s) 120 can analyze the sound signals for an anomaly (e.g., as indicated by changes in the sound signals). For example, processor(s) 120 can perform a spectrum/frequency analysis (e.g., a power analysis) of the heart and/or lungs. The spectrum of the heart and lung sound recordings can be analyzed separately. Based on the sound signals, processor(s) 120 can automatically generate an alert. For example, the alert can indicate that the recipient is healthy or that an anomaly was detected. In one specific example, processor(s) 120 can automatically diagnose the recipient with a medical condition, e.g., by including an indication of the diagnosis with the alert. Alternatively, processor(s) 120 can provide the sound signals to an external device for diagnosis of the recipient with the medical condition. In other words, the medical diagnosis can occur autonomously at implantable medical device 100, at an external device automatically, at an external device (e.g., one that includes a screen to display the sound signals), by a medical professional, or any combination thereof.

Implantable medical device 100 can further include an electrode (e.g., a recording electrode) configured to measure a biopotential signal of the recipient (e.g., as part of a cardiac electrogram (EGM)). In one example, the biopotential signal can cause implantable medical device 100 to generate the notification. Processor(s) 120 can prompt the recipient to produce the particular biological environment based on the biopotential signal of the recipient. As a result, instead of continuously monitoring the heart and lung sounds for diagnostic purposes, implantable medical device 100 can use the biopotential measurements (e.g., when the biopotential signal exceeds a predefined potential variation) to activate/trigger the heart and lung diagnostic procedure.

In another example, the biopotential signal can be correlated with the detected sound signals. In this case, the electrode can measure the biopotential signal of the recipient while implantable microphone 130 is detecting the one or more sound signals associated with the target bodily function of the recipient. Processor(s) 120 can correlate the biopotential signal of the recipient with the one or more sound signals associated with the target bodily function of the recipient. The biopotentials recorded during the sound recordings can provide diagnostic information that relates to heart and lung diagnostics (e.g., the shape of the biopotential recording), and can also be informative for other physiological parameters (e.g., tissue conductivity) when there is no correlation between the increase/decrease of the recorded sound and biopotentials amplitudes.

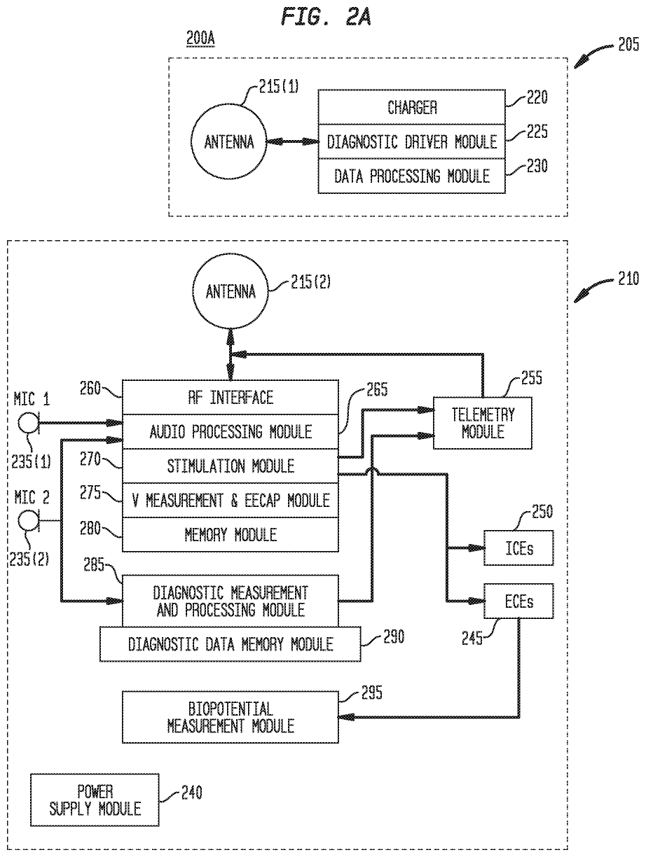

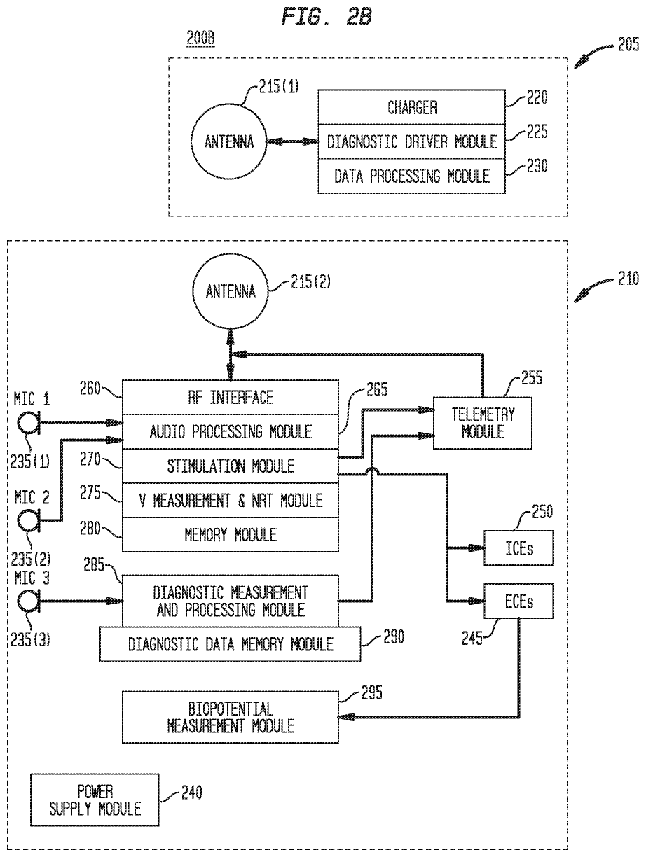

FIG. 2A is a block diagram illustrating a cochlear implant system 200A with which aspects of the techniques presented herein can be implemented. More specifically, cochlear implant system 200A is configured to perform functions associated with cochlear stimulation and to independently perform functions for conducting biological diagnoses as described herein.

The cochlear implant system 200A comprises an external component 205 and an implantable/internal component (e.g., cochlear implant) 210. External component 205 is configured to be directly or indirectly attached to the body of the recipient and implantable component 210 configured to be implanted in the recipient. External component 205 includes antenna 215(1), charger 220, diagnostic driver module 225, and data processing module 230. Internal component 210 includes antenna 215(2), microphones 235(1) and 235(2), power supply module 240, Extra-Cochlear Electrodes (ECEs) 245, Intra-Cochlear Electrodes (ICEs) 250, and telemetry module 255. Internal component 210 further includes an RF interface 260, audio processing module 265, stimulation module 270, voltage and electrically evoked compound action potential (EECAP) module 275, and memory module 280. Internal component still further includes diagnostic measurement and processing module 285, diagnostic data memory module 290, and biopotential measurement module 295.

Antennas 215(1) and 215(2) can transmit data between the external component 205 and the cochlear implant 210 via a closely-coupled wireless link formed between the antennas 215(1) and 215(2). In certain examples, the closely-coupled wireless link is a radio frequency (RF) link. However, various other types of energy transfer, such as infrared (IR), electromagnetic, capacitive and inductive transfer, can be used to transfer the power and/or data from external component 205 to cochlear implant 210. Antennas 215(1) and 215(2) can also enable external component 205 to transmit power to cochlear implant 210. For example, charger 220 can include a charging coil configured to charge power supply module 240, which can include at least one rechargeable battery.

Microphones 235(1) and 235(1) can each be disposed in, or electrically connected to, the implant body of cochlear implant 210. In operation, the microphones 235(1) and 235(2) detect input (sound/vibration) signals (e.g., external acoustic sounds and/or body noises) and convert the detected input signals into electrical signals. Microphones 235(1) and 235(2) can begin collecting the input signals after charger 220 provides test instructions to the recipient. These electrical signals are received by audio processing module 265, which is configured to execute signal processing and coding to convert the electrical signals into processed signals that represent the detected signals. The processed signals are then provided to the stimulation module 270, which is configured to utilize the processed signals to generate electrical stimulation signals via ECEs 245 and ICEs 250. ECEs 245 can include one or more electrodes outside of the cochlea, and ICEs 250 can include a plurality of longitudinally spaced intra-cochlear electrical stimulating contacts (electrodes) that collectively form a contact or electrode array for delivery of electrical stimulation (current) to the recipient's cochlea. In this way, cochlear implant 210 stimulates the recipient's auditory nerve cells, bypassing absent or defective hair cells that normally transduce acoustic vibrations into neural activity. Voltage and EECAP module 275 can collect feedback regarding how the recipient responds to the electrical stimulation, and that feedback can be stored in memory module 280.

One of microphones 235(1) and 235(2) is more sensitive to body noises than it is to external acoustic sound signals. In the illustrative embodiment of FIG. 2A, the implantable microphone 235(1) is a “sound” sensor/transducer that is primarily configured to detect/receive external acoustic sounds (e.g., an implantable microphone), while the implantable microphone 235(2) is a “vibration” sensor that is primarily configured to detect/receive internal body noises for noise cancelation. Microphone 235(1) is affected by body noises that can be characterized as an acceleration coming from the body (or the recipient's own voice) and captured by the microphone 235(1). The cochlear implant 210 uses the microphone 235(2) to control the vibrations and deliver a useful signal to the recipient. That is, in general, the output of the microphone 235(2) is used to cancel/attenuate body noises appearing in the output of the microphone 235(1).

It is to be appreciated that an implantable microphone could have various locations with a recipient. For example, the implantable microphones can be implanted subcutaneously (e.g., beneath the skin/tissue of a recipient), within middle ear cavity of the recipient, etc. It is also be appreciated that, although certain embodiments are described herein with reference to implantable microphones, it is to be appreciated that certain aspects can also be implemented with non-implantable microphones. For example, certain aspects presented herein can be implemented with microphones located within the oral cavity of the recipient, in the ear canal of a recipient, worn by the recipient, etc.

In addition, it is to be appreciated the techniques presented herein can be implemented with a variety of microphone technologies used to detect acoustic sounds and/or vibrations. For example, the microphones used with various embodiments presented herein can be devi electret microphones, electromechanical microphones, piezoelectric microphones, micro-electromechanical system (MEMS) microphones, accelerometers, etc. In certain examples, aspects can be implemented with optical interferometers, pressure sensors, or any other device now known or later developed.

Diagnostic driver module 225 and data processing module 230 can comprise, for example, one or more processors and a memory device (memory) that includes logic for automated diagnosis and processing of data. In operation, diagnostic driver module 225 can prompt the recipient to create a biological environment suitable for taking the measurement (e.g., by instructing the recipient to breathe deeply). In the biological environment (e.g., while the recipient is breathing deeply), microphone 235(2) can detect one or more bodily noises (e.g., a heartbeat) of the recipient and provide bodily noise data (e.g., heartbeat data) to diagnostic measurement and processing module 285.

Diagnostic measurement and processing module 285 can be similar to diagnostic measurement and processing module 150. Diagnostic measurement and processing module 285 can process the bodily noise data, and diagnostic data memory module 290 can store the raw and/or processed bodily noise data and/or pre-recorded bodily noise data (e.g., for cardiac and lung audio signals) that can be used as reference for processing/analysis of the bodily noise data. Diagnostic measurement and processing module 285 can further provide the processed bodily noise data to telemetry module 255, which can also obtain sound data from stimulation module 270. Telemetry module 255 can provide the processed bodily noise data (and/or data obtained from stimulation module 270) via antennas 215(1) and 215(2) to data processing module 230. Data processing module 230 can, in turn, analyze data obtained from telemetry module 255 for potential diagnostic issues (e.g., heart problems).

ECEs 145 can measure biopotential data (e.g., biopotential signals created by cardiac and lung activity). In one example, the biopotentials can be measured simultaneously with the bodily noise data recorded by microphone 235(2) and correlated therewith. In another example, the biopotentials can be used to determine the recording window (e.g., when microphone 235(2) should begin recording the bodily noise data). To prevent the stimulation electrical pulses generated by stimulation module 270 from interfering with the biopotential data, cochlear stimulation can be paused for the duration of the test. It will be appreciated that the recipient can interrupt the test at any point to restore stimulation/hearing. ECEs 145 can provide the biopotential data to biopotential measurement module 295 so that the biopotential data can be correlated with the bodily noise data. The data can be correlated automatically or semi-automatically by cochlear implant system 200A.

FIG. 2B is a block diagram illustrating a cochlear implant system 200B with which aspects of the techniques presented herein can be implemented. Cochlear implant system 200B is similar to cochlear implant system 200A (FIG. 2A), except that cochlear implant system 200B includes an additional microphone 235(3). Microphone 235(3) is a dedicated microphone for diagnostic measurements, while microphones 235(1) and 235(2) are dedicated microphones for providing sound input that is used to stimulate ICEs 250. That is, microphone 235(3) can be configured to obtain the audio data for diagnosis, and microphones 235(1) and 235(2) can be configured to monitor interfering bodily noise to be removed from the audio data obtained by microphone 235(3).

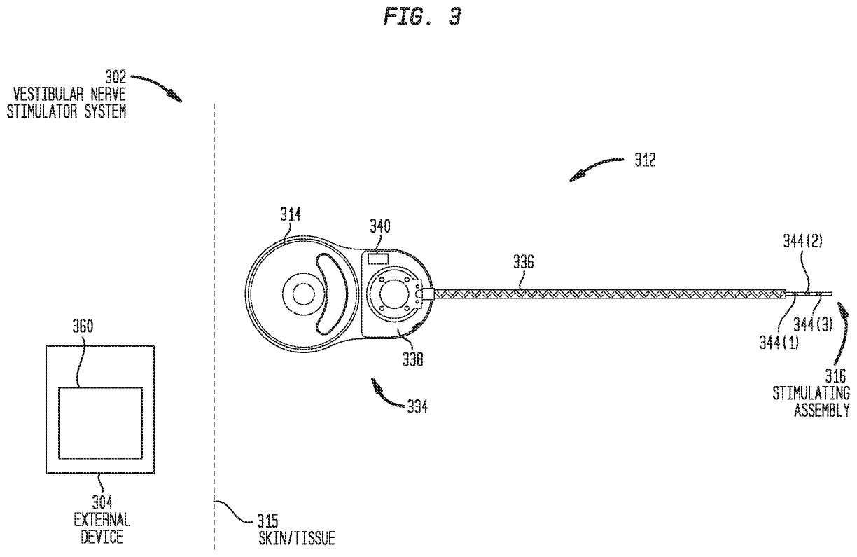

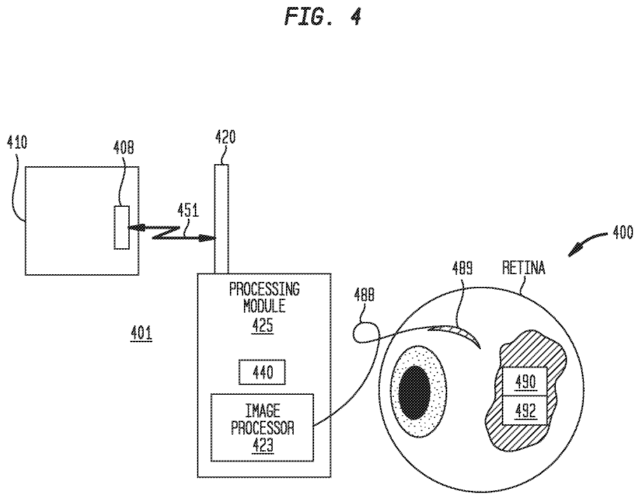

As previously described, the technology disclosed herein can be applied in any of a variety of circumstances and with a variety of different devices. Example devices that can benefit from technology disclosed herein are described in more detail in FIGS. 3 and 4, below. The techniques of the present disclosure can be applied to other medical devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue. Further, technology described herein can also be applied to consumer devices. These different systems and devices can benefit from the technology described herein.

FIG. 3 illustrates an example vestibular stimulator system 302, with which embodiments presented herein can be implemented. As shown, the vestibular stimulator system 302 comprises an implantable component (vestibular stimulator) 312 and an external device/component 304 (e.g., external processing device, battery charger, remote control, etc.). The external device 304 comprises a transceiver unit 360. As such, the external device 304 is configured to transfer data (and potentially power) to the vestibular stimulator 312.

The vestibular stimulator 312 comprises an implant body (main module) 334, a lead region 336, and a stimulating assembly 316, all configured to be implanted under the skin/tissue (tissue) 315 of the recipient. The implant body 334 generally comprises a hermetically-sealed housing 338 in which RF interface circuitry, one or more rechargeable batteries, one or more processors, and a stimulator unit are disposed. The implant body 334 also includes an internal/implantable coil 314 that is generally external to the hermetically-sealed housing 338, but which is connected to the transceiver via a hermetic feedthrough (not shown). In this example, the implant body 334 includes an implantable microphone 340.

The stimulating assembly 316 comprises a plurality of electrodes 344(1)-(3) disposed in a carrier member (e.g., a flexible silicone body). In this specific example, the stimulating assembly 316 comprises three (3) stimulation electrodes, referred to as stimulation electrodes 344(1), 344(2), and 344(3). The stimulation electrodes 344(1), 344(2), and 344(3) function as an electrical interface for delivery of electrical stimulation signals to the recipient's vestibular system.

The stimulating assembly 316 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein can be used with stimulating assemblies having different numbers of stimulation electrodes, stimulating assemblies having different lengths, etc.

As noted, the vestibular stimulator 312 includes an implantable microphone 340. In certain embodiments presented herein, the vestibular stimulator system 302 (e.g., vestibular stimulator 312 and/or external device 304) can operate, as described above, to measure a sound signal from the output of the implantable microphone 340 during a biological state deliberately created by a recipient in response to a prompt from vestibular stimulator system 302. That is, the vestibular stimulator system 302 can include functionality as described above with reference to FIGS. 1, 2A, and 2B.

FIG. 4 illustrates a retinal prosthesis system 401 that comprises an external device 410 configured to communicate with a retinal prosthesis 400 via signals 451. The retinal prosthesis 400 comprises an implanted processing module 425 and a retinal prosthesis sensor-stimulator 490 is positioned proximate the retina of a recipient. The external device 410 and the processing module 425 can communicate via coils 408, 420.

In an example, sensory inputs (e.g., photons entering the eye) are absorbed by a microelectronic array of the sensor-stimulator 490 that is hybridized to a glass piece 492 including, for example, an embedded array of microwires. The glass can have a curved surface that conforms to the inner radius of the retina. The sensor-stimulator 490 can include a microelectronic imaging device that can be made of thin silicon containing integrated circuitry that convert the incident photons to an electronic charge.

The processing module 425 includes an image processor 423 that is in signal communication with the sensor-stimulator 490 via, for example, a lead 488 which extends through surgical incision 489 formed in the eye wall. In other examples, processing module 425 is in wireless communication with the sensor-stimulator 490. The image processor 423 processes the input into the sensor-stimulator 490, and provides control signals back to the sensor-stimulator 490 so the device can provide an output to the optic nerve. That said, in an alternate example, the processing is executed by a component proximate to, or integrated with, the sensor-stimulator 490. The electric charge resulting from the conversion of the incident photons is converted to a proportional amount of electronic current which is input to a nearby retinal cell layer. The cells fire and a signal is sent to the optic nerve, thus inducing a sight perception.

The processing module 425 can be implanted in the recipient and function by communicating with the external device 410, such as a behind-the-ear unit, a pair of eyeglasses, etc. The external device 410 can include an external light/image capture device (e.g., located in/on a behind-the-ear device or a pair of glasses, etc.), while, as noted above, in some examples, the sensor-stimulator 490 captures light/images, which sensor-stimulator is implanted in the recipient.

As noted, the processing module 425 includes an implantable microphone 440. In certain embodiments presented herein, the retinal prosthesis system 401 (e.g., retinal prosthesis 400 and/or external device 410) can operate, as described above, to measure a sound signal from the output of the implantable microphone 440 during a biological state deliberately created by a recipient in response to a prompt from retinal prosthesis system 401. That is, the retinal prosthesis system 401 can include functionality as described above with reference to FIGS. 1, 2A, and 2B.

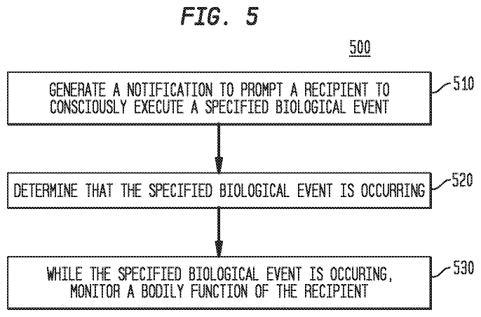

FIG. 5 is a flowchart of an example method 500, in accordance with certain embodiments presented herein. At operation 510, an implantable medical device generates a notification to prompt a recipient to consciously execute a specified biological event. At operation 520, the implantable medical device determines that the specified biological event is occurring. At operation 530, while the specified biological event is occurring, the implantable medical device monitors a bodily function of the recipient.

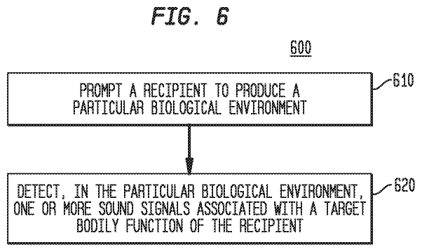

FIG. 6 is a flowchart of an example method 600, in accordance with certain embodiments presented herein. At operation 610, one or more processors of an implantable medical device prompt a recipient to produce a particular biological environment. At operation 620, an implantable microphone of the implantable medical device detects, in the particular biological environment, one or more sound signals associated with a target bodily function of the recipient.

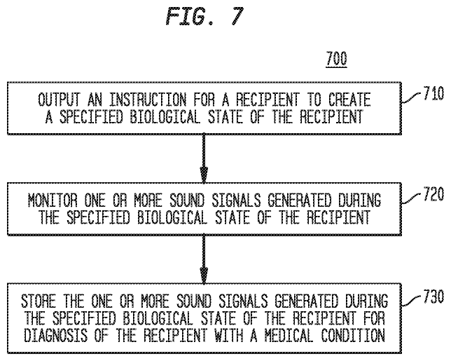

FIG. 7 is a flowchart of an example method 700, in accordance with certain embodiments presented herein. At operation 710, an implantable medical device outputs an instruction for a recipient to create a specified biological state of the recipient. At operation 720, the implantable medical device monitors one or more sound signals generated during the specified biological state of the recipient. At operation 730, the implantable medical device stores the one or more sound signals generated during the specified biological state of the recipient for diagnosis of the recipient with a medical condition.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments can be combined with another in any of a number of different manners.

What is claimed is:

1. A method, comprising:
generating a notification to prompt a recipient to consciously execute a specified biological event;
determining that the specified biological event is occurring; and
while the specified biological event is occurring, monitoring a bodily function of the recipient using at least one implantable sensor.

2. The method of claim 1, wherein generating the notification to prompt the recipient to consciously execute the specified biological event includes:
generating a notification to prompt the recipient to temporarily cease breathing.

3. The method of claim 2, wherein monitoring the bodily function of the recipient includes:
recording at least one of lung activity or heart activity of the recipient.

4. (canceled)

5. (canceled)

6. The method of claim 3, wherein recording the lung activity of the recipient includes:
recording a sound signal while the recipient is breathing deeply; and
subtracting, from the sound signal, a sound signal corresponding to heart activity of the recipient.

7. The method of claim 1, further comprising:
generating the notification to prompt the recipient to consciously execute the specified biological event in response to obtaining, from the recipient, an indication to monitor the bodily function of the recipient.

8. The method of claim 1, further comprising:
generating the notification to prompt the recipient to consciously execute the specified biological event in response to one or more triggering biological events of the recipient.

9. The method of claim 1, further comprising:
generating the notification to prompt the recipient to consciously execute the specified biological event at a predetermined time.

10. The method of claim 1, wherein generating the notification to prompt the recipient to consciously execute the specified biological event includes:
providing at least one of a visual prompt or an auditory prompt for the recipient to consciously execute the specified biological event.

11. (canceled)

12. The method of claim 1, wherein determining that the specified biological event is occurring includes:
determining that a predefined period of time after generating the notification to prompt the recipient to consciously execute the specified biological event has expired, or
obtaining, from the recipient, an explicit indication that the recipient has executed the specified biological event.

13. (canceled)

14. The method of claim 1, wherein determining that the specified biological event is occurring includes:
obtaining a biological indication that the recipient has executed the specified biological event.

15. A medical device comprising:
one or more processors configured to prompt a recipient to produce a particular biological environment; and
a microphone configured to detect, in the particular biological environment, one or more sound signals associated with a target bodily function of the recipient.

16. (canceled)

17. (canceled)

18. (canceled)

19. The medical device of claim 15, further comprising:
an electrode configured to measure a biopotential signal of the recipient.

20. The medical device of claim 19, wherein the one or more processors are configured to prompt the recipient to produce the particular biological environment based on the biopotential signal of the recipient.

21. The medical device of claim 19, wherein the electrode is configured to measure the biopotential signal of the recipient while the microphone is detecting the one or more sound signals associated with the target bodily function of the recipient.

22. The medical device of claim 21, wherein the one or more processors are further configured to correlate the biopotential signal of the recipient with the one or more sound signals associated with the target bodily function of the recipient.

23. (canceled)

24. One or more non-transitory computer readable storage media comprising instructions that, when executed by a processor, cause the processor to:
output an instruction for a recipient to create a specified biological state of the recipient;
monitor one or more sound signals generated during the specified biological state of the recipient; and
store the one or more sound signals generated during the specified biological state of the recipient for diagnosis of the recipient with a medical condition.

25. The one or more non-transitory computer readable storage media of claim 24, further comprising instructions executable to:
compare the one or more sound signals generated during the specified biological state of the recipient with one or more historical sound signals generated during the specified biological state of the recipient.

26. The one or more non-transitory computer readable storage media of claim 24, further comprising instructions executable to:
analyze the one or more sound signals generated during the specified biological state of the recipient for an anomaly.

27. The one or more non-transitory computer readable storage media of claim 24, further comprising instructions executable to:
based on the one or more sound signals generated during the specified biological state of the recipient, automatically generate an alert.

28. The one or more non-transitory computer readable storage media of claim 27, wherein the instructions operable to automatically generate the alert include instructions executable to:
automatically diagnose the recipient with the medical condition.

29-36. (canceled)
